SEQUENCE LISTING (34 sequences)

SEQ ID NO: 1 - DNA, 21 bp, Artificial sequence
  Synthetic
cgctctagaactagtggatcc

SEQ ID NO: 2 - DNA, 20 bp, Artificial sequence
  Synthetic
tcgaggtcgacggtatcgat

SEQ ID NO: 3 - DNA, 29 bp, Artificial sequence
  Synthetic
acacctgatcaagccgttcatttgattac

SEQ ID NO: 4 - DNA, 25 bp, Artificial sequence
  Synthetic
cgccaaagaatgatctgcggagctt

SEQ ID NO: 5 - DNA, 51 bp, Artificial sequence
  Synthetic
cgctctagaactagtggatccacacctgatcaagcctgttcatttgattac

SEQ ID NO: 6 - DNA, 45 bp, Artificial sequence
  Synthetic
tcgaggtcgacggtatcgatcgccaaagaatgatctgcggagctt

SEQ ID NO: 7 - DNA, 46 bp, Artificial sequence
  Synthetic
cgctctagaactagtggatccacactttatgcttccggctcgtatg

SEQ ID NO: 8 - DNA, 42 bp, Artificial sequence
  Synthetic
tcgaggtcgacggtatcgataacgttcttcggggcgaaaact

SEQ ID NO: 9 - DNA, 633 bp, Artificial sequence
  Synthetic
acacctgatcaagcctgttcatttgattaccagagagactgtcatgatccacatggagggaaggacatgtgtgttgctggagccattcaaaatttcacatctcagcttggccatttccgccatggaacatctgatcgtcgatataatatgacagaggctttgttatttttatcccacttcatgggagatattcatcagcctatgcatgttggatttacaagtgatatgggaggaaacagtatagatttgcgctggtttcgccacaaatccaacctgcaccatgtttgggatagagagattattcttacagctgcagcagattaccatggtaaggatatgcactctctcctacaagacatacagaggaactttacagagggtagttggttgcaagatgttgaatcctggaaggaatgtgatgatatctctactagcgccaataagtatgctaaggagagtataaaactagcctgtaactggggttacaaagatgttgaatctggcgaaactctgtcagataaatacttcaacacaagaatgccaattgtcatgaaacggatagctcagggtggaatccgtttatccatgattttgaaccgagttcttggaagctccgcagatcattctttggcg

SEQ ID NO: 10 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 11 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcggattgttgggccctgaattgaaaaaggaagagtatgagtattcaacatttccgtgtcgcccttattccctttcttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 12 - DNA, 585 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattaactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattccctttttttgcggcattttgccttcccgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 13 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaagcgaattcgcggccgctaaattcaattcgccctatagtgagtcgtattacaattcactggccgtcgttttacaacgtcgtgaccgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 14 - DNA, 585 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 15 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaggagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 16 - DNA, 584 bp, Artificial sequence; identical to SEQ ID NO: 10
  Synthetic

SEQ ID NO: 17 - DNA, 584 bp, Artificial sequence; identical to SEQ ID NO: 10
  Synthetic

SEQ ID NO: 18 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacttaatcgccttgcagcacttccccctttcgccaggtggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 19 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgaccgggaaaaccctggcgttacccaacttaatcgccttgcagcacatccccctttcgccagctggcgtaatagcgaagaggcccgcaccgatcgcccttcccaacagttgcgcagcctatatgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 20 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccctattcccttttttgcggcattttgccttcctgcttttgctcacccagaaacgctggtgaaagtaaaagatgctgaagatcagttaggtgcacgagtgggttacatcgaactggatctcaacagcggtaagatccttgagagttttcgccccgaagaacgttttcgcgcggc

SEQ ID NO: 21 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagcgagtcgtattacaattcactggccgtcgttttacaacgtcgtgactgggaaaactctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 22 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagtcgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagaggtaagatccttgagagttttcgccccgaagaacgttttcgcgcggc

SEQ ID NO: 23 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacctaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatctccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 24 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaatccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 25 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcagttcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 26 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctatattcaattcgccctatagtgagtcgtattacaattcactggccgtcgttttacaacgtcgtgaccgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaagggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 27 - DNA, 584 bp, Artificial sequence
  Synthetic
acactttatgcttccggctcgtatgttgtgtggaattgtgagcggataacaatttcacacaggaaacagctatgaccatgattacgccaagctcagaataaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 28 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggttaaaacgaattcgcgtccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 29 - DNA, 584 bp, Artificial sequence
  Synthetic
acactttatgcttccggctcgtatgttgtgtggaattgtgagcggataacaatttcacacaggaaacagctatgaccatgattacgccaagctcagaattatccctcactaaagggactagtcctgcaggtttaaacgaattcgcggccgctaaattcaattcgccctaaagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 30 - DNA, 584 bp, Artificial sequence
  Synthetic
acactttatgcttccggctcgtatgttgtgtggaattgtgagcggataacaatttcacacaggaaacagctatgactatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgctacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 31 - DNA, 584 bp, Artificial sequence
  Synthetic
acactttatgcttccggctcgtatgttgtgtggaattgtgagcggataacaatttcacacaggaaacagctatgaccatgattacgccaagctcagaattgaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttactcccttttttgcggcactttgccttcctgtttttgctcacccagaaacgctggtgaaagcaaaagatgctgaagatcagttgggtgcacgagtgggttacatcgaactggatctcaacagcggtaagatccttgagagttttcgccccgaagaacgttttcgcgcggc

SEQ ID NO: 32 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgcttcacaacgtcgtgactgggaaaaccctggcgttacccaacttaatcgccttgcagcacatccccctttcgccagccggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 33 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctacaggtttaaacgaattcgcggccgctaaattcaattcgccctatagtgagttgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacgcaagggcaaatcgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

SEQ ID NO: 34 - DNA, 584 bp, Artificial sequence
  Synthetic
acactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcggccgctaaattcaattcgccctatagtgagtcgtattacaattcactggccgtcgttttacaacgtcgtgactgggaaaaccctggcgttacccaacctaatcgccttgcagcacatccccctttcgccagctggcgtaatagcgaagaggcccgcaccgatcgcccttcccaacagttgcgcagcctatacgtacgcaagggcgaattgttgggccctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttcgcgcggc

(19) United States
(12) Patent Application Publication
(10) Pub. No.: US 2008/0113354 A1
(43) Pub. Date: May 15, 2008

(54) Methods and Compositions for High Sensitivity Fluorescent Mutation Detection with Mismatch Cutting Dna Endonucleases

(76) Inventors: Yanggu Shi, Gaithersburg, MD (US); Gary F. Gerard, Frederick, MD (US)

Correspondence Address: LICATA & TYRRELL P.C., 66 E. MAIN STREET, MARLTON, NJ, 08053, US

(21) Appl. No.: 11/718,761

(22) PCT Filed: Nov. 14, 2005
(86) PCT No.: PCT/US05/41056

§ 371 Date: Aug. 23, 2007

Related U.S. Application Data
(60) Provisional application No. 60/627,609, filed on Nov. 12, 2004.

(51) Int. Cl. C12Q 1/68; C07H 21/04

(52) U.S. Cl. 435/6; 536/24.33

(57) ABSTRACT

Methods and kits are provided with DNA substrates having a fluorescent label positioned at a nucleotide internal from its 5′ end for use with CEL nuclease to determine whether a DNA sequence contains mutations or polymorphic changes.

This patent application claims the benefit of priority from U.S. Provisional Application Ser. No. 60/627,609, filed Nov. 12, 2004, teachings of which are herein incorporated by reference in their entirety.

### FIELD OF THE INVENTION

The present invention relates to improved methods to fluorescently label DNA substrates for use with CEL nuclease and other mismatch cutting DNA endonucleases to determine whether a DNA sequence contains mutations or polymorphic changes. The present invention also relates to a one-step universal fluorescent PCR primer technique to generate fluorescent PCR products for enzymatic mutation detection by CEL nuclease and other mismatch cutting DNA endonucleases. These methodologies provide for highly sensitive, high throughput, economic mismatch detection in all DNA samples.

### BACKGROUND OF THE INVENTION

The accurate and efficient detection of both inherited and induced mutations in genomes is a critical step for the diagnosis of diseases and drug discovery. A highly sensitive, high throughput and economic mutation detection technique is essential for these areas of endeavor. There have been a number of methodologies developed for mutation detection; all have limitations that restrict their uses.

A novel family of DNA mismatch-specific endonucleases from plants was discovered recently (Oleykowski et al. Nucl. Acid Res. 1998 26:4597-4602; Yang et al. Biochem. 2000 39:3533-3541). The plant source with the highest apparent concentration of this class of endonucleases is celery (Oleykowski et al. Nucl. Acid Res. 1998 26:4597-4602), and thus the enzyme was purified from celery and named CEL I (Oleykowski et al. Nucl. Acid Res. 1998 26:4597-4602; Yang et al. Biochem. 2000 39:3533-3541). CEL I cleaves DNA at the 3′-side of sites of base-substitution mismatch and DNA distortion (Oleykowski et al. Nucl. Acid Res. 1998 26:4597-4602; Yang et al. Biochem. 2000 39:3533-3541).

Purified preparations of CEL nuclease identified as CEL I actually contain two different protein species (Yang et al. Biochem. 2000 39:3533-3541; U.S. Pat. No. 5,869,245). One species, called CEL I, has been purified and characterized and its gene has been sequenced and cloned (Yang et al. Biochem. 2000 39:3533-3541; U.S. Pat. No. 5,869,245). CEL I nuclease has been used to accurately detect a variety of mutations and polymorphisms in the human BRCA1 gene (Oleykowski et al. Nucl. Acid Res. 1998 26:4597-4602; Yang et al. Biochem. 2000 39:3533-3541; Kulinski et al. BioTechniques 2000 29:44-48). The second protein species present in purified preparations of CEL I, called CEL II, has been separated from CEL I, purified and characterized, and its gene has been sequenced and cloned. CEL II has been used to verify the presence of known mutations in a number of genes in human peripheral blood DNA (Scaffino et al. Transgenics 2004 4:157-166), to carry out screening for induced point mutations in barley (Caldwell et al. The Plant Journal 2004 doi:10.111/j.1365-313X.204.02190.x), to screen for error-free clones generated from a plant cDNA library by PCR-based cloning (Qiu et al. Molecular Biotechnology 2005 29:11-18), and to screen for mutations in the mitochondrial DNA of patients with respiratory chain defects (Bannwarth et al. Human Mutation 2005 25:575-582).

CEL I nuclease and CEL II nuclease have a unique enzymatic property that has been demonstrated advantageous in mutation detection (Oleykowski et al. Nucl. Acid Res. 1998 26:4597-4602; Yang et al. Biochem. 2000 39:3533-3541; Kulinski et al. BioTechniques 2000 29:44-48; Colbert et al. Plant Physiology 2001 126:480-484; Sokurenko et al. Nucl. Acids. Res. 2001 29:e11; U.S. Pat. No. 5,869,245; Qiu et al. BioTechniques 2004 36:702-707). With a DNA duplex containing mismatches such as a substitution, insertion or deletion, CEL nuclease cleaves the mismatched structure to generate DNA fragments which can be identified with gel electrophoresis, HPLC or capillary electrophoresis detection platforms. Compared to primer extension methods, CEL nuclease mutation detection requires no prior knowledge of the position or the nature of the mutation. In heterogeneous DNA samples, such as in somatic mutations and heteroplasmy, a CEL nuclease-based method outperforms direct sequencing where base calling is difficult or impossible. Moreover, the ability to pool DNA samples in CEL nuclease mutation detection significantly increases the throughput for large population samples, while at the same time reduces associated costs.

Fluorescent labeling of DNA samples offers major benefits to CEL nuclease mutation detection methods including increased signal intensity relative to ultraviolet (UV) absorbance for detection of DNA, reduced sample quantity requirement for application to high throughput polyacrylamide or capillary electrophoretic instruments suited to automated detection and data collection and handling, and multicolor/multichannel capability with selected fluorescent dyes for sample pooling and increased dependability in data analysis.

One of the enzymatic characteristics of the CEL nuclease family of plant DNA endonucleases is the tendency to remove nucleotides from the 5′ ends of double-stranded DNA molecules. Unfortunately this tendency to remove DNA 5′-end nucleotides reduces the sensitivity of detection of DNA labeled at the 5′ end with a fluorophore. Replacing phosphate-oxygen groups with phosphate-sulfur groups at internucleoside linkages near the DNA 5′ end does not prevent the hydrolysis. Thus, the amount of CEL nuclease relative to 5′-end labeled DNA substrate must be carefully controlled in a reaction so that the amount of residual fluorescent signal remains sufficient for fluorescent capillary electrophoresis detection after CEL nuclease mismatch cutting.

### SUMMARY OF THE INVENTION

In the present invention, a method to determine mutations and/or polymorphic changes in DNA sequences via CEL nuclease is provided wherein a fluorescent label is positioned at a nucleotide internal from the 5′ end of a double-stranded DNA thereby protecting the label from CEL exonuclease removal. It has now been found that by placing a fluorescent dye on a base downstream from the 5′ end of double-stranded DNA, greater than 90% of the label is preserved during CEL nuclease treatment. Based upon this finding, internally labeled fluorescent PCR primers have now been produced to amplify target DNA sequences for subsequent CEL nuclease mutation detection. With these primers and using capillary electrophoresis detection, the signal was dramatically increased and mutations could be detected at a level of 1% in a wild-type DNA population. As demonstrated herein, these primers and methodologies for use are useful not only with CEL nuclease but other mismatch cutting endonucleases such as KAL III.

Thus, one aspect of the present invention relates to the design and use of internally labeled fluorescent PCR primers for generating fluorescent PCR products in mismatch cutting DNA endonuclease mutation detection.

Another aspect of the present invention relates to the design and use of internally labeled fluorescent universal nucleotide sequences as universal fluorescent PCR primers and unlabeled primers containing the universal primer sequence in one-step or separate PCR reactions, as a means to generate fluorescent PCR products, for mismatch cutting DNA endonuclease nuclease mutation detection.

Another aspect of the present invention relates to methods for carrying out PCR amplifications in a one-step PCR reaction or a nested PCR reaction using these PCR primers.

Another aspect of the present invention relates to methods of incorporating fluorescent labels internally into DNA molecules.

Another aspect of the present invention relates to methods for placing fluorescent dyes, either identical or distinct, on both ends of a DNA molecule.

Another aspect of the present invention relates to methods for pooling labeled DNA samples for multiplexed mutation detection to increase detection throughput and reduce assay costs.

Another aspect of the present invention relates to methods for enzymatic digestion by CEL nuclease or other mismatch cutting nucleases of labeled DNA products for detecting the presence, position and nature, in the nucleotide sequence, of mutations or sequence variations.

Another aspect of the present invention relates to methods for fluorescence detection of the labeled DNA products by capillary electrophoresis, gel electrophoresis, HPLC, and other fluorometric methods.

Another aspect of the present invention relates to kits for carrying out the methods of the present invention. In one embodiment, the kit comprises universal fluorescent PCR primers containing the same or different fluorescent dyes and sequence information of the universal priming sites. Kits of the present invention may further comprise rules for designing target sequence specific PCR primers for embedding internal fluorescent dye in PCR products by universal fluorescence priming, PCR DNA polymerase and related PCR reaction components, and/or CEL nuclease and/or related buffers as well as primer sets specific for target sequence(s) of interest.

### BRIEF DESCRIPTION OF THE DRAWINGS

FIG. 1 is a thin layer chromatogram showing mono-, di- and trinucleotides released from a 64 basepair DNA duplex terminally labeled at one 5′ end with ³²P by the exonuclease activity of CEL II nuclease at various time points. Lane −E: no CEL II nuclease present.

FIGS. 2A and 2B depict an agarose gel from experiments demonstrating that an internal fluorescent label is protected from CEL II nuclease removal. Control G PCR products were unlabeled (Lane 1 and 2); were labeled at the 5′ end with 6-FAM (Lane 3 and 4); or were labeled internally near the 5′ end with fluorescein (Lane 5 and 6). The DNAs (200 ng) were treated with 5 units of CEL II nuclease at 42° C. for 20 minutes (Lane 2, 4, and 6) or with buffer alone (Lane 1, 3, 5). The panel in FIG. 2A depicts darkreader fluorescent imaging showing fluorescent end label; the panel in FIG. 2B shows UV imaging of total DNA stained with the intercalating dye ethidium bromide.

FIGS. 3A and 3B are schematic drawings of internally labeled synthetic universal fluorescent primers FKS (fluorescein labeled universal primer; FIG. 3A; SEQ ID NO:1) and TSK (TAMPA labeled universal primer; FIG. 3B; SEQ ID NO:2).

FIG. 4 is a schematic diagram of exemplary one-step and nested fluorescent PCR amplification techniques performed in accordance with the present invention. In this diagram, (G1) is the gene specific forward priming sequence, (U1) is the universal priming site, (G2) is the gene specific reverse priming sequence, (U2) is the universal priming site, (F1) is the fluorescent universal primer with the same sequence as (U1), and (F2) is the fluorescent universal primer with the same sequence as (U2).

FIGS. 5A and 5B is an agarose gel depicted PCR incorporation of universal primer labeled with fluorescein and mixed at different ratios with the unlabeled primer KS.CELR. Primer mixtures of labeled to unlabeled primer of 0:1, 1:1, 9:1, 19:1 were used in one-step amplification/labeling PCR reactions. The total concentration of the combined FKS and KS.CELR primers was 0.5 μM. The PCR products were separated by electrophoresis on a 3% agarose gel. The panel depicted in FIG. 5A is from darkreader fluorescent imaging showing fluorescent end labeled DNA. The panel depicted in FIG. 5B shows UV imaging of the total DNA stained with ethidium bromide.

FIG. 6 shows capillary electrophoresis chromatograms of Control G homoduplex and Control G/C heteroduplexes digested with CEL II nuclease. Control G and C DNAs were labeled by PCR amplification with universal primers FKS downstream and FCELF upstream; labeled Control G and C DNAs were annealed at different ratios; 200 ng of total DNA was digested with 5 units of CEL II nuclease at 42° C. for 20 minutes; and the digestion products were separated by capillary electrophoresis on an ABI PRISM® 3100 Genetic Analyzer. The percentages indicate the amounts of Control C in Control C/G heteroduplex DNA.

FIG. 7 shows capillary electrophoresis chromatograms of PCR amplified Lac Z mutant DNAs annealed with amplified wild-type DNA and digested with CEL II. The PCR amplification products of 26 different Lac Z mutant plasmid DNAs labeled with primer FKS were annealed separately with amplified wild-type DNA, digested with 5 units of CEL II nuclease at 42° C. for 20 minutes, and separated by capillary electrophoresis on an ABI PRISM® 3100 Genetic Analyzer. The digestion fragment sizes for clones 1-6 are indicated and the sizes agree with those predicted based upon CEL II nuclease cleavage at the location of mutations determined by DNA sequencing (see Appendix 2).

FIGS. 8A, 8B and 8C provide a comparison of DNA sample processing methods after CEL II nuclease digestion. Fluorescent tag-labeled Control C/G heteroduplex DNAs at different ratios of Control C to Control G were digested with CEL II nuclease. The samples were prepared in HiDi loading solution without prior processing (straight loading (FIG. 8A)), with prior ethanol precipitation to remove salt in the sample (FIG. 8B), or with prior desalting on Microspin G-25 columns (FIG. 8C). The Control C/G heteroduplex DNA for straight loading and ethanol precipitation was labeled at one end with FKS primer. The Control C/G heteroduplex DNA processed by desalting on Microspin G-25 columns was labeled at both ends by the use of an internal fluorescein modified forward PCR primer, FCELF (5′-ACACCTGATCAAGCC[FdT]GTTCATTTGATTAC-3′ (SEQ ID NO:3), 411-bp fragment) and FKS (232-bp fragment).

FIG. 9 shows results from experiments detecting a LacZ mutation with mismatch cutting enzyme KAL III. Sample processing was performed in accordance with procedures outlined in FIG. 7 except that DNAs were cleaved with KAL III nuclease.

### DETAILED DESCRIPTION OF THE INVENTION

CEL nuclease specifically cuts DNA mismatches including single-base substitutions, deletions, and insertions in a DNA duplex. Such cleavage produces DNA fragments indicative of mutation(s) between wild-type reference and mutant DNA.

Fluorescent labeling of the 5′ ends of DNA samples with one or more fluorophores can greatly increase detection sensitivity and sample throughput when coupled with an appropriate fractionation/detection platform. However, because CEL nuclease also possesses exonuclease activity that efficiently removes nucleotides at DNA 5′ ends, fluorescent label at DNA 5′ends added by use of conventionally synthesized PCR primers is rapidly removed by CEL nuclease, thus diminishing detection sensitivity.

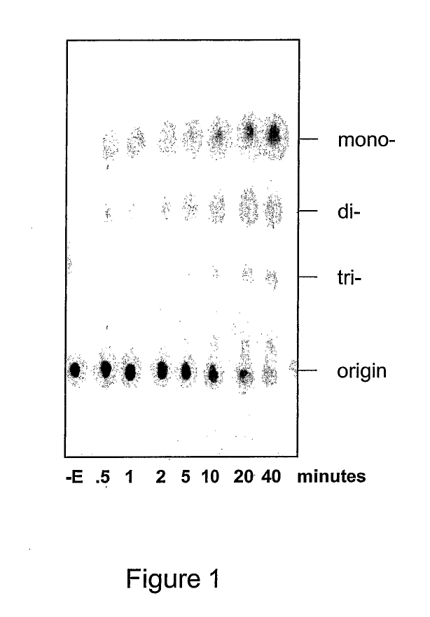

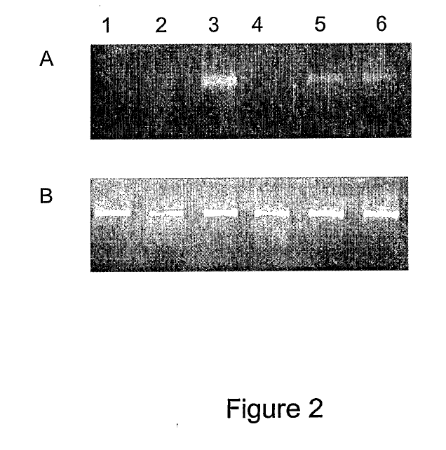

The efficient exonuclease activity that removes nucleotides from the 5′ ends of double-stranded DNA is shown in FIG. 1. In this experiment, a 64-base synthetic oligonucleotide labeled at the 5′ end with ³²P with polynucleotide kinase and annealed to an unlabeled complementary 64 mer was incubated with 5 units of CEL II nuclease at 42° C. for 0.5, 1, 2, 5, 10, 20, 40 minutes and analyzed by thin layer chromatography. ³²P-Labeled mono- and dinucleotides were immediately released within 0.5 and 2 minutes. At the 20-minute time point, a typical incubation time for CEL II nuclease enzymatic mutation detection, the majority of the ³²P label migrated as mononucleotide and to a lesser extent as dinucleotide. The exonuclease activity of CEL II nuclease and members of this family presents a significant problem for well-established and convenient methods used to fluorescently label DNA molecules at 5′ ends for CEL nuclease mutation detection. In fact, as shown by experiments depicted in FIG. 2, PCR product with 5′ end 6-FAM label (see FIG. 2, Lane 3) lost most of its fluorescence after CEL II digestion and became invisible on an agarose gel when fluorescence from the 6-FAM was measured (FIG. 2, Lane 4).

The present invention overcomes this problem by placing a fluorescent label on a nucleotide base of a PCR primer internal to the 5′ end. For example, FIG. 2 provides results from experiments comparing CEL II digestion using a 5′ labeled PCR primer versus a PCR primer with a fluorescein label placed 16 bases internally in a PCR primer. In these experiments, digestion with CEL II was performed at 42° C. for 20 minutes. In contrast to 5′ end fluorescent label (FIG. 2, Lanes 3 and 4), the internal label was well preserved (FIG. 2, Lane 5 and Lane 6). Densitometry showed that internal labeling resulted in retention of 97% of the label after CEL II nuclease digestion (mean intensity 49.68 vs. 50.18, CEL II digested vs. undigested). These results indicate that CEL II exonuclease activity is confined to removal of a few bases from the 5′ end of double-stranded DNA in the 20 minute incubation. This understanding was utilized to synthesize fluorescently labeled substrates, and in particular PCR primers resistant to removal of label by CEL II nuclease.

Accordingly, one aspect of the present invention relates to PCR primers useful in DNA mutation detection assays via CEL nuclease or other mismatch cutting DNA endonucleases which comprise a PCR primer labeled at a nucleotide internal to the 5′ end of the PCR primer. By internal to the 5′ end it is meant that the label, preferably a fluorescent label, is place on a nucleotide base of the primer at least 4, more preferably at least 7, even more preferably at least 10 nucleotide bases away from the 5′ end. Examples of such fluorescent dyes or labels useful in these primers include, but are not limited to 6-FAM, fluorescein, TAMRA, HEX, NED, ROX, rhodamines, JOE, Cy3, Cy5, Texas Red, and Alexa fluorescent dyes.

The present invention also provides a method for universal PCR amplification/fluorescence labeling using common universal fluorescent PCR primers labeled in this fashion for any target gene and universal PCR primers produced thereby. Examples of methods for incorporating fluorescent labels internally into DNA molecules include, but are in no way limited to, the use of polymerases, terminal deoxynucleotide transferases, or ligases to incorporate internal labels for the purpose of preserving the labels from removal by CEL nuclease and other mismatch cutting DNA endonucleases.

The method and universal primers offer advantages to preparing individual labeled PCR primers for each target gene. These advantages include significant cost reduction in having to prepare only two labeled primers rather than individual labeled primer pairs for each target, shorter turn around time to prepare PCR primers, prequalified and consistent universal fluorescent primers to avoid the variability in signal intensity associated with the use of individual primers labeled internally at different positions.

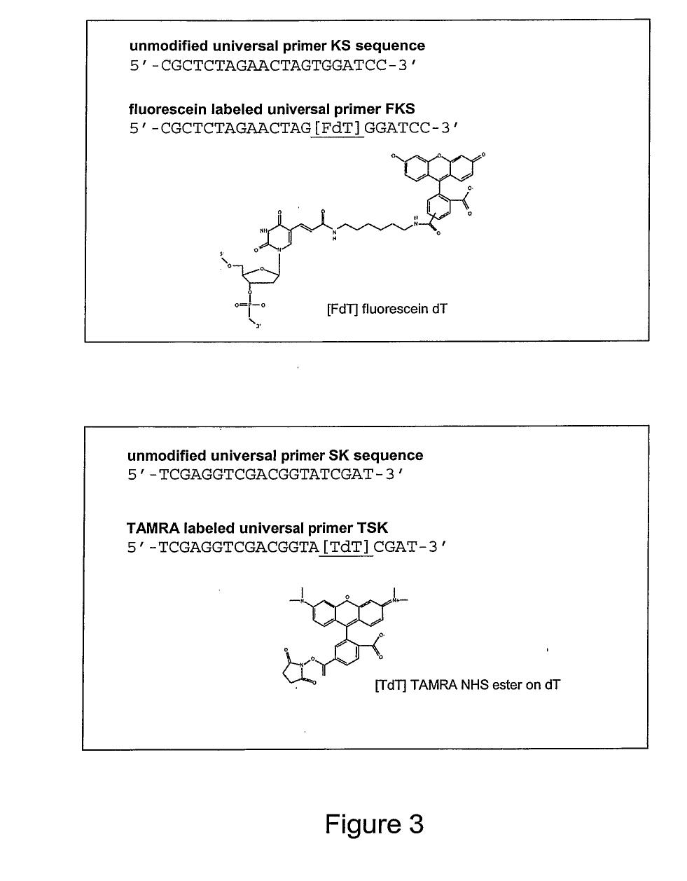

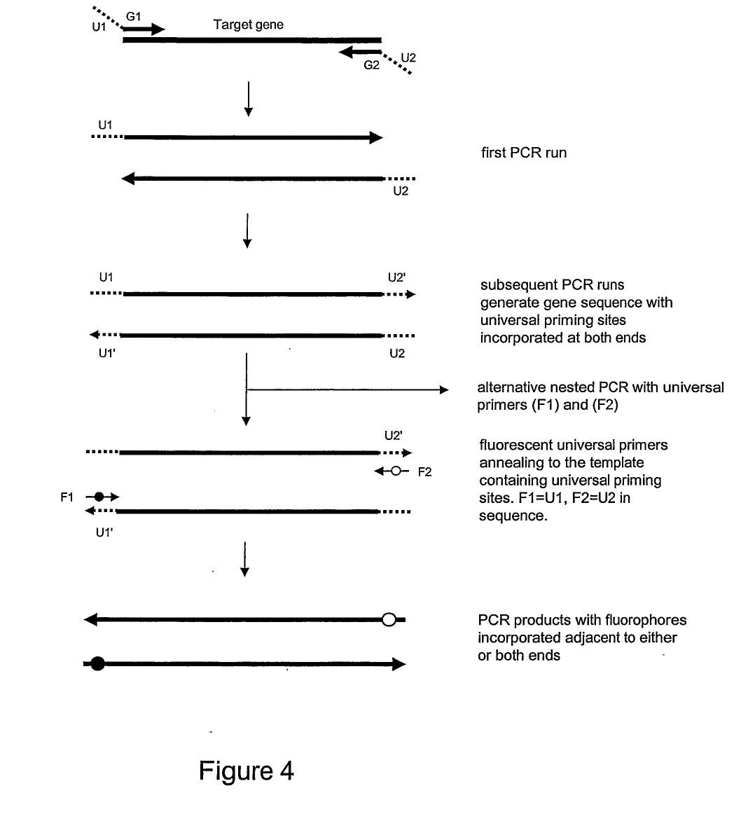

Exemplary universal primers of the present invention are depicted in FIG. 3. As shown in FIG. 3, universal primers with SK and KS sequences, as examples, are internally labeled with fluorophores, such as fluorescein and TAMRA. To generate fluorescent PCR product of a given gene, regular PCR primers are synthesized that include the SK or KS sequence as a universal priming site at the 5′ end. For example, primer pairs 5′-ACACCTGATCAAGCCTGTTCATTTGATTAC-3′ (SEQ ID NO:3) and 5′-CGCCAAAGAATGATCTGCGGAGCTT-3′ (SEQ ID NO:4) for regular PCR are synthesized as 5′-[CGCTCTAGAACTAGTGGATCC]ACACCTGATCAAGCCTGTTCATTTGATTAC-3′ (SEQ ID NO:5) and 5′-[TCGAGGTCGACGGTATCGAT]CGCCAAAGAATGATCTGCGGAGCTT-3′ (SEQ ID NO:6). Either one or both of the universal fluorescent primers (TSK or FKS) can be used as outlined in FIG. 4 to generate fluorescent PCR product for CEL nuclease mutation detection.

FIG. 4 sets forth an exemplary PCR reaction performed with primers of the present invention, wherein the target gene as the template is annealed with 0.05 μM forward primer and 0.05 μM reverse primer, in which (G1) is the gene specific forward priming sequence, (U1) is the universal priming site, (G2) is the gene specific reverse priming sequence, (U2) is the universal priming site, (F1) is the fluorescent universal primer with the same sequence as (U1), and (F2) is the fluorescent universal primer with the same sequence as (U2). In this example, (G1) and (G2) have a calculated Tm equal to or greater than 60° C. Further, in this example, (F1) and (F2) contain internally labeled fluorophores such as TSK or FKS described in FIG. 3. For single reaction amplification/fluorescent labeling PCR depicted in this exemplary Figure, 0.5 μM fluorescent universal primers (F1) and/or (F2) are included. After 14 cycles of PCR at an annealing temperature of 60° C., the amplicon is amplified at 55° C. for additional 20 cycles. The 10-fold excess of fluorescent universal primers over unlabeled gene specific primers results in PCR product being labeled efficiently. In an alternative exemplary method, the target gene is first amplified by standard PCR for 30 cycles. The PCR product, preferably 10 ng, is then taken as the template in a separate nested PCR reaction with fluorescent universal primers F1 and F2.

The PCR can be carried out in one reaction or in two-steps similar to nested PCR. In the one-step reaction, the amount of the universal fluorescent primer is in 10-fold excess over the gene specific primer containing the universal priming site. In one embodiment, a single-reaction PCR was carried out with the following PCR cycles:
95° C. for 2 minutes
14 cycles of
   95° C. for 30 seconds
   60° C. for 30 seconds
   72° C. for 1.5 minutes
20 cycles of
   95° C. for 30 seconds
   55° C. for 30 seconds
   72° C. for 1.5 minutes
   72° C. for 5 minutes
4° C. until use.

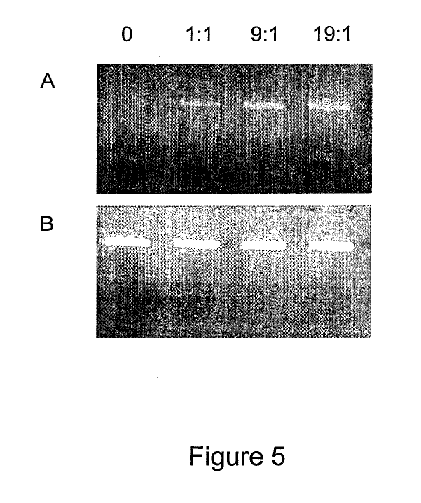

The labeling efficiency and product yield of this exemplary single-step PCR reaction of the present invention are displayed in FIG. 5. The optimal ratio of universal fluorescent primer to gene specific primer was 9:1. The higher universal fluorescent primer to gene specific primer molar ratio (19:1) did not increase the fluorescence significantly and might reduce the reliability for a more complex DNA template such as genomic DNA.

As understood by the skilled artisan upon reading this disclosure, the number of cycles used in the first round of PCR is not limited to 14 cycles as exemplified herein, and a larger number of cycles may be required for a more complex DNA template such as a genomic DNA. Similarly, the skilled artisan will understand upon reading this disclosure that the number of cycles used in the second round of single-reaction PCR is not limited to 20 cycles as exemplified, but rather can be varied depending upon the yield of labeled PCR product desired. A preferred yield is at least 40 ng/μl of PCR reaction mixture.

Accordingly, another aspect of the present invention relates to a single reaction amplification/fluorescent labeling polymerase chain reaction (PCR) which comprises a plurality of cycles at an annealing temperature with primers of the present invention, preferably at least 14 cycles, followed by a plurality of cycles of amplification, preferably a sufficient number of cycles to produce a yield of 40 ng/μl of PCR reaction mixture. In an alternative embodiment, the present invention relates to an amplification/fluorescent labeling nested polymerase chain reaction (PCR) comprising amplifying a target gene by standard PCR and using the resulting PCR product as a template in a separate PCR reaction with primers of the present invention.

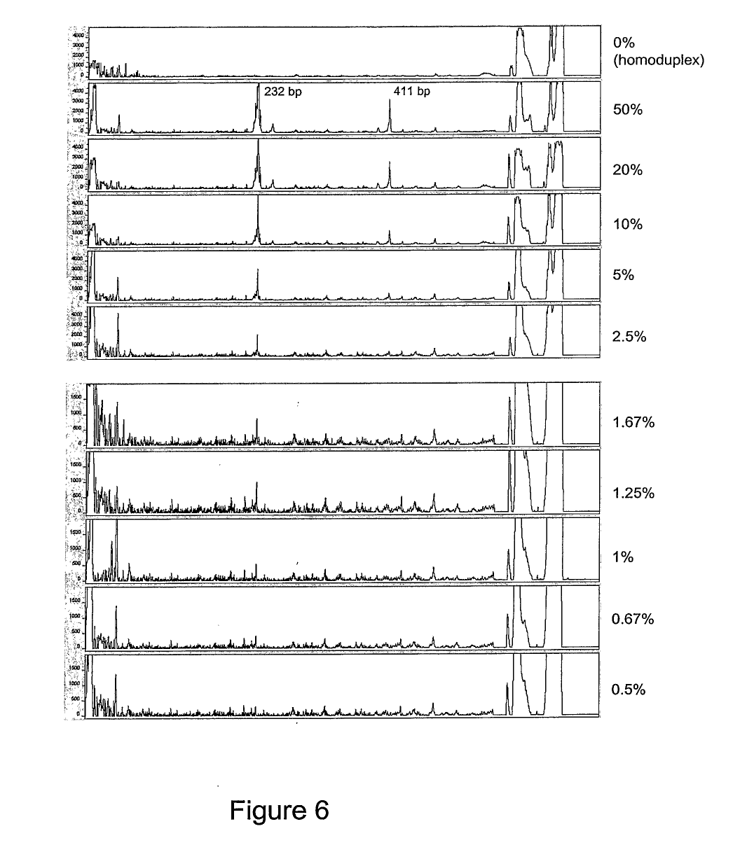

Using the primers and methodologies of the present invention, two 633 bp DNA sequences (Control G and Control C; see Appendix 1) with one G>C basepair change were amplified and fluorescently labeled by single-reaction PCR. The size of the PCR products was increased to 653 bp as the universal priming KS sequence was included at the downstream end. Control G DNA was annealed to itself (homoduplex) or with decreasing amounts of Control C (heteroduplex). The total amount of the DNA used as substrate was constant at 200 ng in a 5-μl reaction volume. The DNAs were annealed in 1×PCR buffer at 95° C. for 2 minutes, 95° C. to 85° C. cooling at −2° C./minute, 85° C. to 25° C. at −0.2° C./minute. Each of the DNA samples was digested with 5 units of CEL II nuclease incubated at 42° C. for 20 minutes and the reaction was stopped by addition of 1 μl 0.5 M EDTA. The digests were precipitated with 2.5 volume of ethanol and resuspended in 10 μl of HiDi solution containing ROX size standard. The samples were subjected to capillary electrophoresis analysis on an ABI PRISM 3100 Genetic Analyzer (see FIG. 6). The cleavage of the mismatch by CEL II nuclease produced two fragments: a 232-bp fragment labeled with FKS and a 411-bp fragment labeled directly with a primer containing an internal fluorescein. Due to the better fluorescein emission quality of the FKS-label, signal from the 232-bp fragment was stronger than that from the 411-bp fragment. Furthermore, the detection limit reached, 1% Control C in Control G, was greatly improved over that observed previously with 5′-end labeled Control G/C heteroduplex DNA substrate (12% detection limit; Qiu et al. BioTechniques 2004 36:702-707).

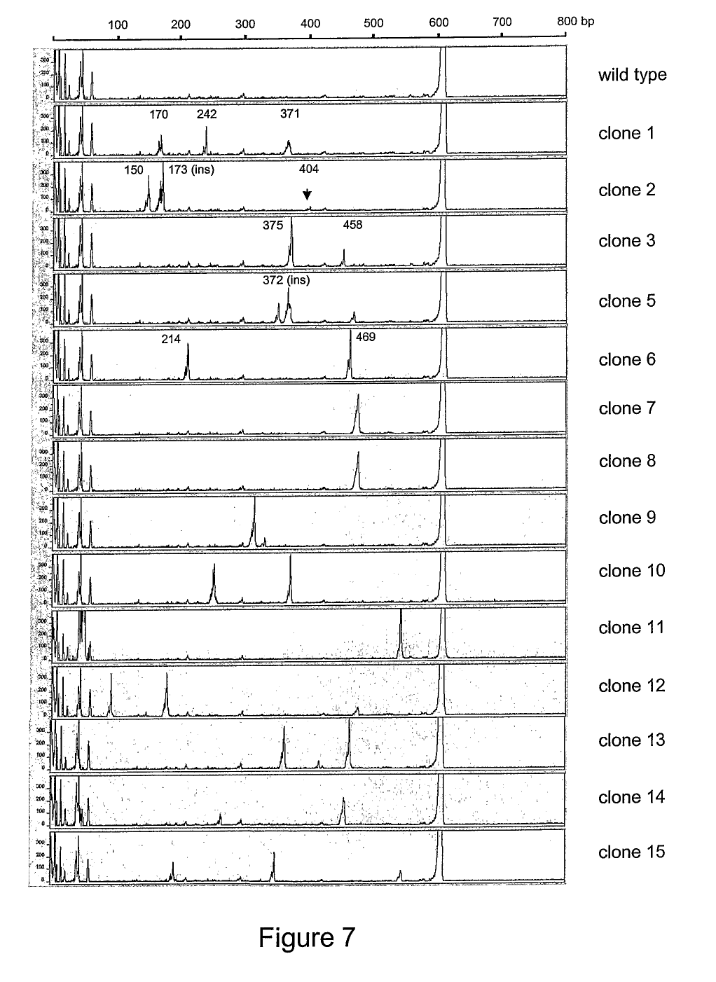

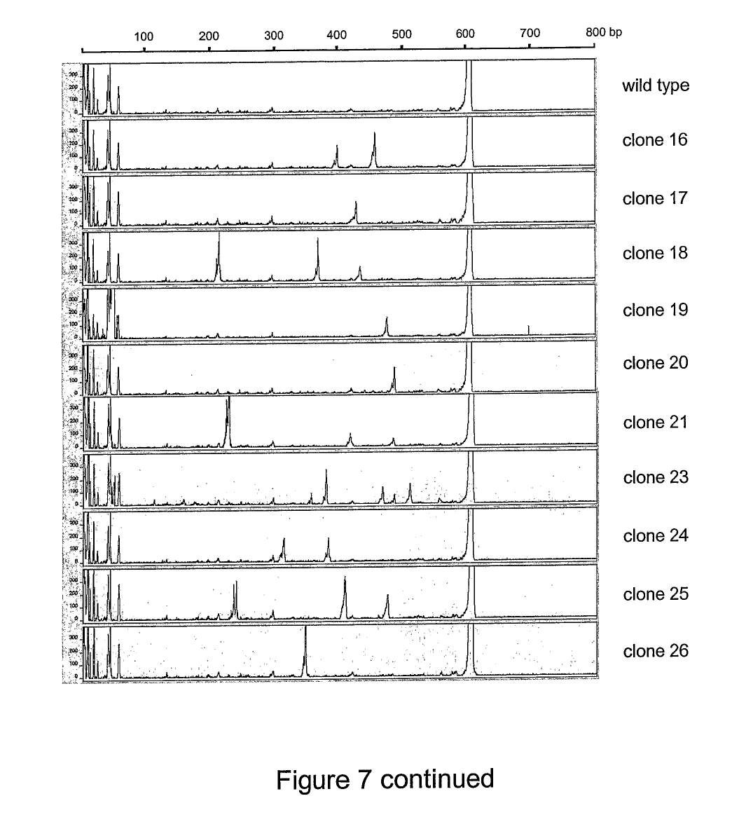

In addition to a G>C substitution in the Control G/C heteroduplex, the primers and methodologies of the present invention were used to examine other mutations including substitutions, insertions, and deletions in a collection of LacZ gene mutants. These LacZ mutants are depicted herein Appendix 2. Gene specific primers used in these experiments were 5′-CGCTCTAGAACTAGTGGATCCACACTTTATGCTTCCGGCTCGTATG-3′ (SEQ ID NO: and 5′-TCGAGGTCGACGGTATCGATAACGTTCTTCGGGGCGAAAACT-3′ (SEQ ID NO:8). FKS was used as the universal fluorescent primer in single reaction PCR. Mutations in Lac Z gene mutant DNAs PCR amplified and labeled in this fashion were correctly identified when digested with CEL II nuclease (see FIG. 7). Digestion of amplified DNAs from clones with multiple mutations produced digestion products of the expected sizes.

It was found that dual fluorescent dye labeling at both ends of Control G/C heteroduplexes with TSK (TAMRA label) and FKS (fluorescein) could be used to detect each fragment produced by CEL II nuclease cutting in separate color channels of fluorescence.

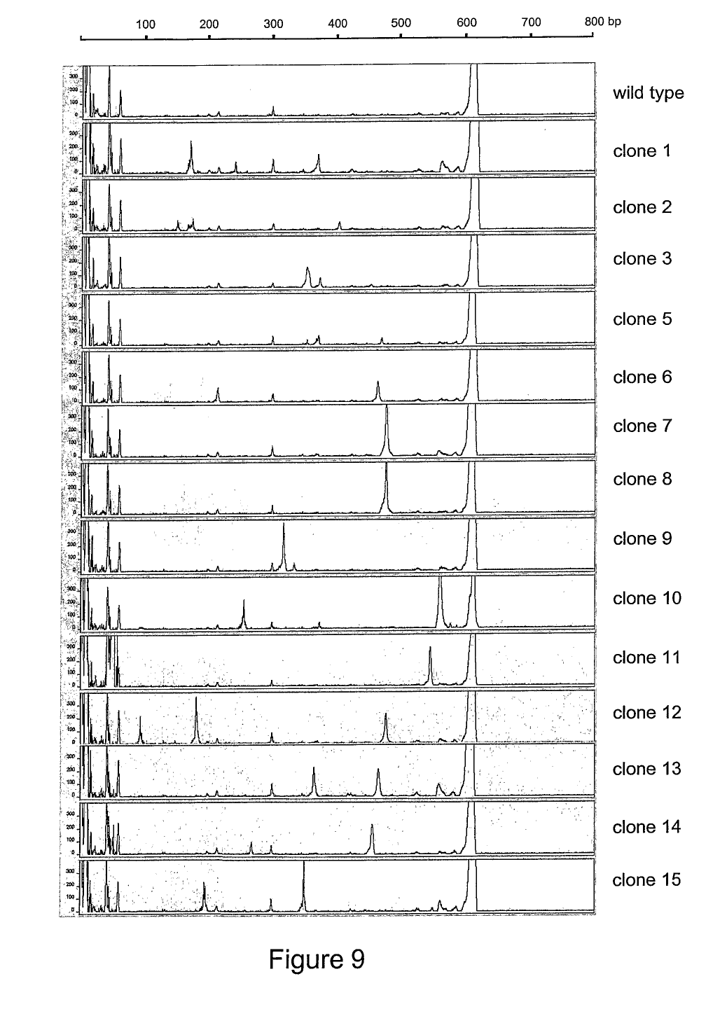

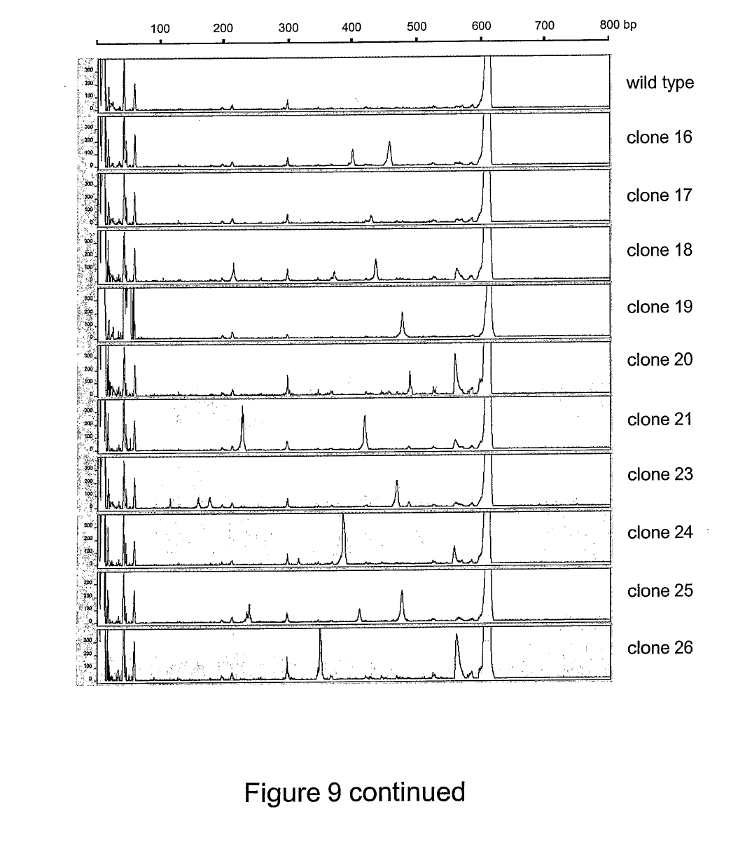

KAL III, isolated from kale, is another mismatch cutting DNA endonuclease similar to CEL II. To demonstrate applicability of the primers and methodologies described herein to other mismatch cutting DNA endonucleases, the DNA duplexes described above were also digested with KAL III Results from this experiment are depicted in FIG. 9. KAL III produced digestion patterns similar to CEL II. Accordingly, the same methods of PCR product labeling and capillary electrophoresis are equally applicable to CEL II and KAL III nuclease and other plant DNA endonucleases similar to CEL II. These experiments are indicative of the primers and methodologies described herein to be useful with other mismatch cutting DNA endonuclease as well including but not limited to other endonucleases of the same family derived from celery, kale and other plants.

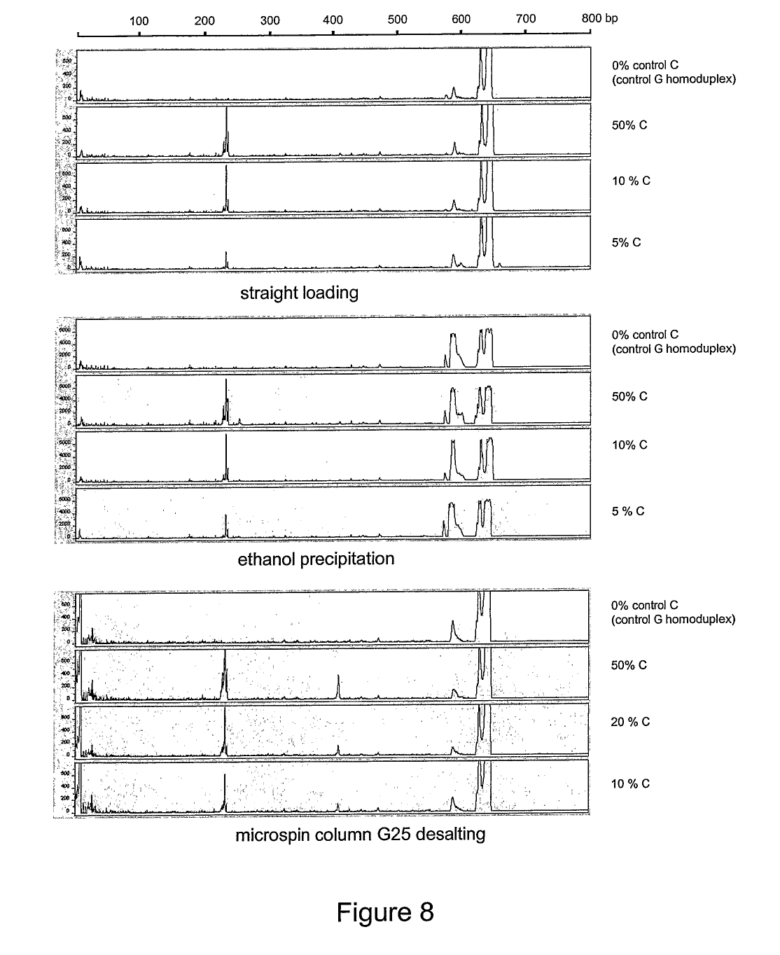

Proper sample processing is critical when high sensitivity mutation detection is desired. For example, one consideration that impacts capillary electrophoresis is that the buffer salt in the samples can interfere with the electrokinetic sample loading. For example, the maximum amount of the sample for straight loading is 1 μl of reaction mixture diluted 10 fold in HiDi loading dye for the ABI PRISM 3100 Genetic Analyzer (FIG. 8, upper panel). Ethanol precipitation serves to remove the salt and concentrate the sample (FIG. 8, middle panel) and produces greater than a 10-fold increase in signal intensity. Gel filtration with a Microspin G-25 column, which removes salt without concentrating the DNA in a reaction mixture, also improves the amount of DNA that is injected and thus the signal strength (FIG. 8, lower panel). Thus, in a preferred embodiment of the methodologies of the present invention, the DNA sample is treated to reduce the salt concentration without concentrating the DNA in the sample.

In preferred embodiments of any of the above methods or kits, universal priming sites are added to the 5′ end of normal PCR primer sequences designed for the amplification of a target sequence in DNA, such as genomic DNA. Amounts (1/10) of forward and reverse primers mixed with universal fluorescent primers (9/10) are included in a one-step amplification/labeling PCR reaction. Alternatively, target DNA can be PCR amplified first with unlabeled primers, and approximately 1% of the PCR reaction is used as the template in a second round of nested PCR with 100% universal fluorescent primers. DNA heteroduplex is formed by hybridization of mutant and wild-type DNA prepared with the methods described. After CEL nuclease digestion, the DNA is analyzed by capillary electrophoresis, such as with ABI PRISM® 3100 Genetic Analyzer. For increased sensitivity, the materials can be desalted by ethanol precipitation or G-25 spin column filtration to aid electrophoretic sample loading.

### APPENDIX 1

### Control G DNA Sequence (SEQ ID NO:9)

The base change from G to C in Control C is underlined.

ACACCTGATCAAGCCTGTTCATTTGATTACCAGAGAGACTGTCATGATCC
ACATGGAGGGAAGGACATGTGTGTTGCTGGAGCCATTCAAAATTTCACAT
CTCAGCTTGGCCATTTCCGCCATGGAACATCTGATCGTCGATATAATATG
ACAGAGGCTTTGTTATTTTTATCCCACTTCATGGGAGATATTCATCAGCC
TATGCATGTTGGATTTACAAGTGATATGGGAGGAAACAGTATAGATTTGC
GCTGGTTTCGCCACAAATCCAACCTGCACCATGTTTGGGATAGAGAGATT
ATTCTTACAGCTGCAGCAGATTACCATGGTAAGGATATGCACTCTCTCCT
ACAAGACATACAGAGGAACTTTACAGAGGGTAGTTGGTTGCAAGATGTTG
AATCCTGGAAGGAATGTGATGATATCTCTACTAGCGCCAATAAGTATGCT
AAGGAGAGTATAAAACTAGCCTGTAACTGGGGTTACAAAGATGTTGAATC
TGGCGAAACTCTGTCAGATAAATACTTCAACACAAGAATGCCAATTGTCA
TGAAACGGATAGCTCAGGGTGGAATCCGTTTATCCATGATTTTGAACCGA
GTTCTTGGAAGCTCCGCAGATCATTCTTTGGCG

### APPENDIX 2

### LacZ Wild Type DNA and Mutant Sequences

Bold indicates the starting point and end point of the amplified region.

Italics indicates a primer sequence.

Lowercase indicates the noncoding region.

Uppercase indicates the coding region.

The start codon is underlined.

Gray highlight indicates a point mutation.

Gray highlight with underline indicates a deletion.

Bold underline indicates an insertion.

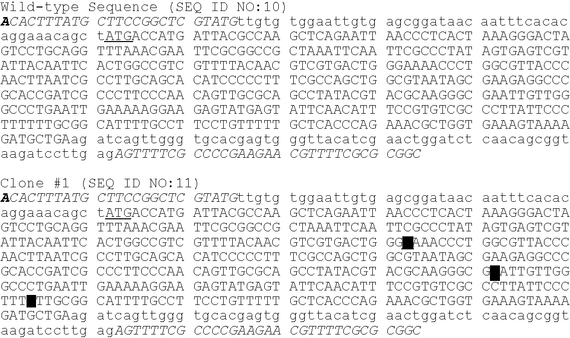

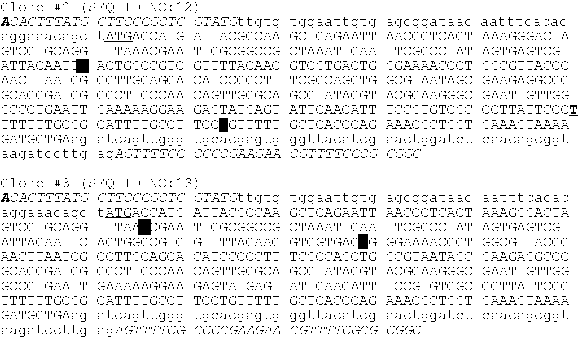

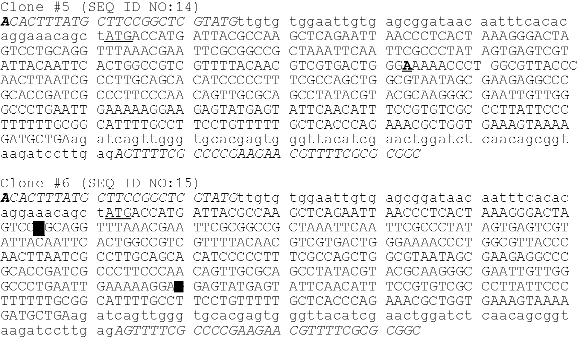

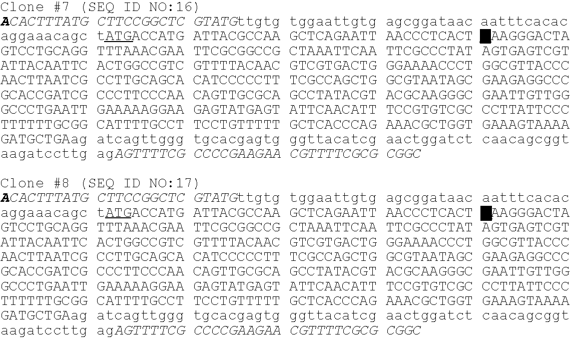

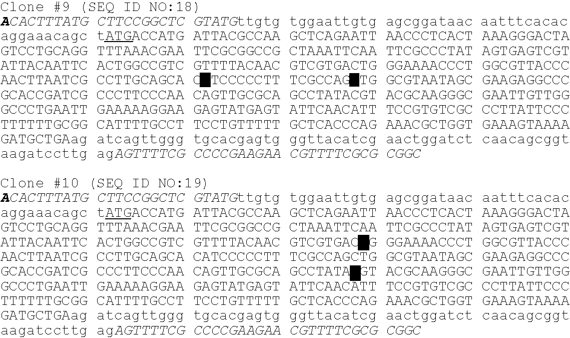

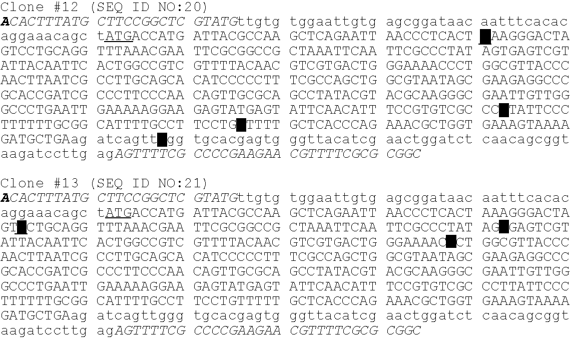

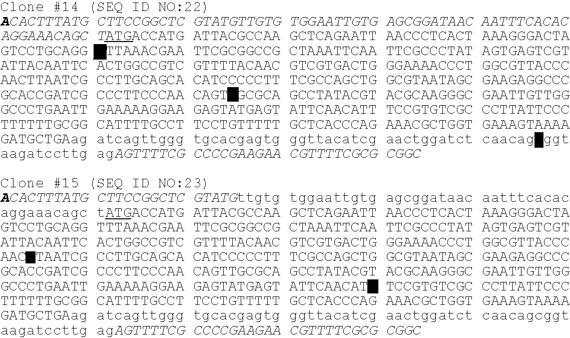

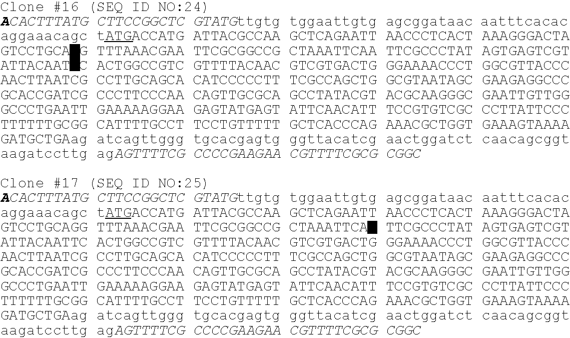

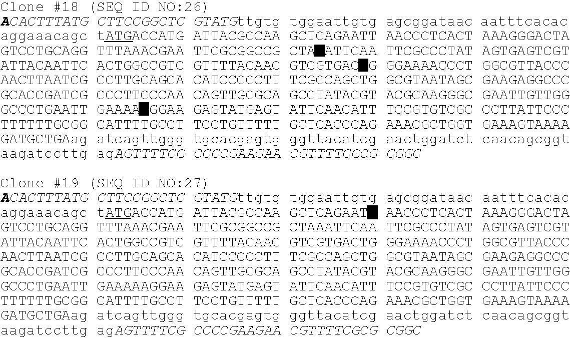

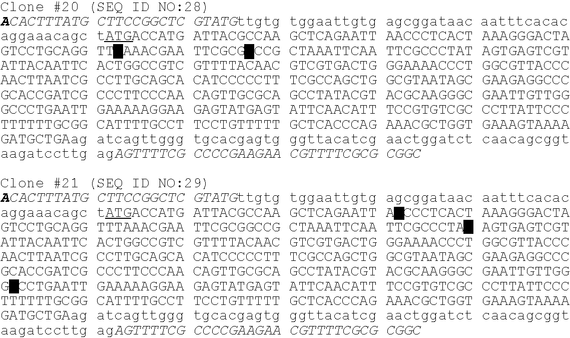

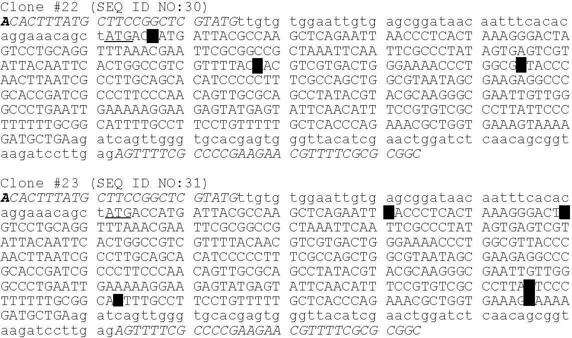

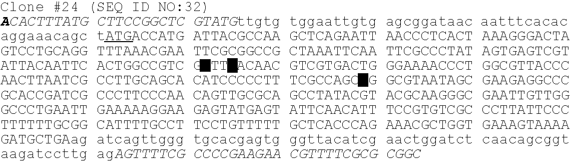

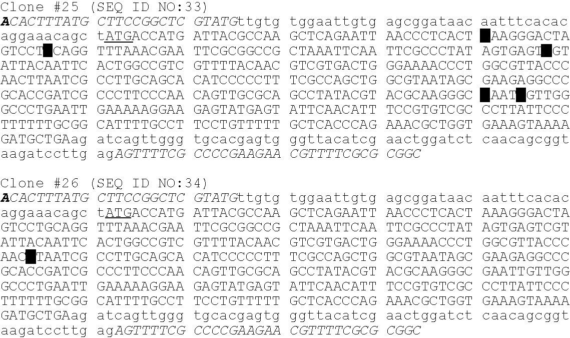

1. A PCR primer for generating fluorescent PCR products in mismatch cutting DNA endonuclease mutation detection, said primer comprising a nucleotide sequence with a 5′ end and 3′ end and a fluorescent label attached to a nucleotide of the nucleotide sequence which is internal with respect to a 5′ end of the nucleotide sequence.

2. The PCR primer of claim 1 wherein the nucleotide sequence is a universal sequence.

3. A single reaction amplification/fluorescent labeling polymerase chain reaction (PCR) comprising a plurality of cycles at an annealing temperature with primers of claim 1 followed by a plurality of cycles of amplification.

4. (canceled)

5. An amplification/fluorescent labeling nested polymerase chain reaction (PCR) comprising amplifying a target gene by standard PCR and using the resulting PCR product as a template in a separate PCR reaction with primers of claim 1.

6. (canceled)

7. A method for detecting mutations in a DNA sequence with a mismatch cutting DNA endonuclease comprising amplifying and fluorescence labeling a DNA sample in accordance with the amplification/fluorescent labeling nested polymerase chain reaction (PCR) of claim 3, digesting the DNA with a mismatch cutting DNA endonuclease; and detecting fluorescently any digested DNA fragments indicative of a mutation in the DNA sequence.

8. The method of claim 7 wherein fluorescence detection is performed by capillary electrophoresis, gel electrophoresis or high pressure liquid chromatography.

9. The method of claim 7 wherein the DNA sample is treated to reduce the salt concentration without concentrating the DNA in the sample prior to detecting fluorescently any digested DNA fragments by capillary electrophoresis.

10. The method of claim 7 wherein the mismatch cutting DNA endonuclease is from the CEL nuclease family of DNA endonucleases.

11. The method of claim 7 wherein the mismatch cutting DNA endonuclease is CEL I nuclease.

12. The method of claim 7 wherein the mismatch cutting DNA endonuclease is CEL II nuclease.

13. A kit for single reaction or nested amplification/fluorescent labeling polymerase chain reaction (PCR) comprising a primer of claim 1.

14. The kit of claim 13 further comprising rules for designing target sequence specific PCR primers for embedding internal fluorescent dye in PCR products by universal fluorescence priming, PCR DNA polymerase or PCR reaction components.

15. A kit for detecting mutations in a DNA sequence with a mismatch cutting DNA endonuclease, said kit comprising a primer of claim 1 and a mismatch cutting DNA endonuclease.

16. The kit of claim 15 wherein the mismatch cutting DNA endonuclease is CEL nuclease.

17. A method for detecting mutations in a DNA sequence with a mismatch cutting DNA endonuclease comprising amplifying and fluorescence labeling a DNA sample in accordance with the amplification/fluorescent labeling nested polymerase chain reaction (PCR) of claim 5, digesting the DNA with a mismatch cutting DNA endonuclease; and detecting fluorescently any digested DNA fragments indicative of a mutation in the DNA sequence.

18. The method of claim 17 wherein fluorescence detection is performed by capillary electrophoresis, gel electrophoresis or high pressure liquid chromatography.

19. The method of claim 17 wherein the DNA sample is treated to reduce the salt concentration without concentrating the DNA in the sample prior to detecting fluorescently any digested DNA fragments by capillary electrophoresis.

20. The method of claim 17 wherein the mismatch cutting DNA endonuclease is from the CEL nuclease family of DNA endonucleases.

21. The method of claim 17 wherein the mismatch cutting DNA endonuclease is CEL I nuclease.

22. The method of claim 17 wherein the mismatch cutting DNA endonuclease is CEL II nuclease.

23. The single reaction amplification/fluorescent labeling polymerase chain reaction (PCR) of claim 3 wherein the nucleotide sequence of the primer is a universal sequence.

24. The single reaction amplification/fluorescent labeling polymerase chain reaction (PCR) of claim 5 wherein the nucleotide sequence of the primer is a universal sequence.

25. A kit for single reaction or nested amplification/fluorescent labeling polymerase chain reaction (PCR) comprising a primer of claim 2.

26. The kit of claim 25 further comprising rules for designing target sequence specific PCR primers for embedding internal fluorescent dye in PCR products by universal fluorescence priming, PCR DNA polymerase or PCR reaction components.

27. A kit for detecting mutations in a DNA sequence with a mismatch cutting DNA endonuclease, said kit comprising a primer of claim 2 and a mismatch cutting DNA endonuclease.

28. The kit of claim 27 wherein the mismatch cutting DNA endonuclease is CEL nuclease.
